(19) United States
(12) Patent Application Publication
(10) Pub. No.: US 2013/0110140 A1
(43) Pub. Date: May 2, 2013

(54) ANASTOMOSIS SYSTEM

(71) Applicant: NATIONAL YANG-MING UNIVERSITY, TAIPEI CITY (TW)

(72) Inventors: Chih-Hsun Lin, Taipei City (TW); Ruey-Yug Tsay, Taipei City (TW)

(73) Assignee: NATIONAL YANG-MING UNIVERSITY, TAIPEI CITY (TW)

(21) Appl. No.: 13/718,209

(22) Filed: Dec. 18, 2012

Related U.S. Application Data
(63) Continuation-in-part of application No. 12/698,477, filed on Feb. 2, 2010, now abandoned.

(30) Foreign Application Priority Data
Oct. 30, 2009 (TW) 098136955

(51) Int. Cl. A61B 17/11

(52) U.S. Cl. CPC A61B 17/11
USPC 606/153

(57) ABSTRACT

An anastomosis system includes a base, two rings, and a blood vessel-leading unit. A first blood vessel can be inserted in and attached to the first ring by the blood vessel-leading unit. A second blood vessel can be inserted in and attached to the second ring by the blood vessel-leading unit. The first ring is placed on a ring-guiding base in a specific manner. The second ring is placed on the ring-guiding base in a movable and rotatable manner to assure the alignment of the blood vessels in both axial and angular directions. The two rings are interconnected by at least one snap-on mechanism or buckle so that the interconnection of the rings is efficient.

### CROSS-REFERENCE

The present application is a continuation-in-part application of U.S. patent application Ser. No. 12/698,477 of which the entire disclosure is incorporated herein for reference.

### BACKGROUND OF THE INVENTION

1. Field of the Invention

The present invention generally relates to an anastomosis system and, more particularly, to an effective and efficient micro-vascular anastomosis system for joining together two blood vessels without having to turning any one of the blood vessel inside out.

2. Description of the Related Art

Anastomosis of blood vessels is one of the greatest achievements in contemporary surgery which expands the surgical field to provide patients with better treatment. Surgical operations such as heart bypass surgery, solid-organ transplantation, radiocephalic fistula and micro-vascular free flap reconstruction are required to be finished with the anastomosis of blood vessels. As for micro-vascular free flap reconstruction, the anastomosis of blood vessels makes it possible for the patients with congenital anomalies, malignant tumor invasion, or injured-caused massive soft tissue defects to receive auto-graft to resurface the defects, and then improve function and appearance.

The present procedure of micro-vascular anastomosis is mainly stitching by hand. However, the outcome of this traditional method for anastomosis of blood vessels relies on the skill and experience of a surgeon. It may therefore result in problems of taking long time, blood leakage from pinhole, and/or blood vessel occlusion caused by stitching opposite sides of the blood vessels or twisting the blood vessels. Hence, there have been quite a few studies for various anastomosis techniques to solve these problems.

In general, there are two types of suture-less anastomosis techniques, one is by chemical and the other one is by physical bonding for tissue fixation. For the one by chemical bonding, it has been disclosed in many literatures to apply tissue glue or laser welding for the anastomosis of blood vessels. However, not any actual clinical applications have been developed from these types of methods because of their complicated and inconvenient operating procedures and the difficulty in maintaining a high patency.

Current clinical implementations of micro-vascular anastomosis techniques are mainly mechanical bonding. Based on their fixation mechanism, these mechanical tissue fixation methods include: stapling, clamping, coupling by a ring-shaped anastomotic coupler. For stapling, a launcher shoots an anastomotic staple to pierce the two ends of blood vessels intended to be anastomosed, and then bends the staple to fix the tissues. For clamping, a vascular clip is applied to hold the two ends of blood vessels to be anastomosed in a non-piercing way. However, because it is difficult to maintain the stress distribution in the blood vessels uniformly, the blood vessels might easily be damaged by this method. To improve this, some ring-shaped couplers are developed in succession.

The fixation mechanisms of a ring-shaped anastomotic coupler are similar to those of stapling and clamping methods. Among them, the “ring-pin” type coupler is a design using stapling as the fixation mechanism and the “SYNOVIS” GEM micro-vascular anastomotic coupler system (SYNOVIS MICRO COMPANIES ALLIANCE, INC. USA) is the only ring-pin type coupler currently on the market. The “extraluminal cuffing ring” is a coupler using clamping as tissue holding mechanism, which does not have any commercialized products yet.

In comparison with the traditional hand-stitched method, the above-mentioned mechanical bonding methods indeed effectively reduce the operation time and the requirements in surgical skills of anastomosis. However, a common drawback of these methods is that the blood vessels have to be everted for 90 degrees or even 180 degrees in operation, which is not applicable to the blood vessels with atherosclerotic change and might cause blood vessel spasm due to tension at the anastomosis site or insufficient blood vessel length for eversion. This drawback has substantially restricted the implementation of these methods in micro-vascular anastomosis. These ring-shaped anastomotic couplers require a complicated staple launcher or alignment equipment which makes the system expensive. Furthermore, the anastomotic staple or ring-pin type anastomotic coupler fixed the tissue by piercing blood vessels, which inevitably damages the blood vessels. The adherence by tissue clips or extraluminal cuffing ring also may cause pressure necrosis of blood vessel walls. Because of the distinct material properties of the anastomotic couplers and the blood vessels, it is likely to cause local compliance mismatch of the blood vessels and disturb the transmission of pulse waves in blood circulation.

As disclosed in U.S. Pat. No. 4,747,407, an anastomosis kit includes a male clamp 5a and a female clamp 5b. A doctor uses the male clamp 5a to clamp an anastomosis wheel 1 and uses the female clamp 5b to clamp another anastomosis wheel 1. Then, the doctor moves the clamps 5a and 5b to each other to bring the anastomosis wheels 1 together. Almost the entire process for bringing the anastomosis wheels 1 together is not guided by any device. Only in a final phase, insertion of bolts 13a in holes 13b is used as means for alignment of the anastomosis wheels 1 with each other. It is however difficult to insert the bolts 13a in the holes 13b because they are small. As being movable relative to each other, the grooves 10 are useless for the alignment of the anastomosis wheels 1 with each other. Moreover, each of the anastomosis wheels 1 includes pins 4 that somehow limit the rotation of an anastomosis wheel 1 relative to the other anastomosis wheel 1 so that one of the blood vessels might be twisted.

The present invention is therefore intended to obviate or at least alleviate the problems encountered in prior art.

### SUMMARY OF THE INVENTION

The primary objective of the present invention is to provide an effective and efficient anastomosis system.

To achieve the foregoing objective, the anastomosis system includes a base, two rings, and a blood vessel-leading unit. A first blood vessel can be inserted in and attached to the first ring by the blood vessel-leading unit. A second blood vessel can be inserted in and attached to the second ring by the blood vessel-leading unit. The first ring is then rested on a ring-guiding base in a specific manner. The second ring is placed on the ring-guiding base in a movable and rotatable manner to assure the alignment of the blood vessels in both axial and angular directions. The two rings are interconnected by at least one snap-on mechanism or buckle.

Other objectives, advantages and features of the present invention will be apparent from the following description referring to the attached drawings.

### BRIEF DESCRIPTION OF THE DRAWINGS

All of the objects, advantages and features of the present invention will become more apparent from the following detailed description taken in conjunction with the accompanying drawings wherein:

FIG. 1 is a cross-sectional view of a blood vessel-leading unit of an anastomosis system in accordance with a first embodiment of the present invention;

FIG. 2 is a perspective view of two rings and a ring-guiding unit of the anastomosis system in accordance with the first embodiment of the present invention;

FIG. 3 is a side view of the rings and the ring-guiding unit shown in FIG. 2;

FIG. 4 is a perspective view of the ring-guiding unit shown in FIG. 2 and two rings of an anastomosis system in accordance with a second embodiment of the present invention;

FIG. 5 is a cross-sectional view of the rings and the ring-guiding unit shown in FIG. 4;

FIG. 6 is a cross-sectional view of the first ring shown in FIG. 5;

FIG. 7 is a front view of the first ring shown in FIG. 6;

FIG. 8 is a cross-sectional view of the second ring and a ring-driving element of the ring-guiding unit shown in FIG. 5;

FIG. 9 is a front view of the second ring and the ring-driving element shown in FIG. 8;

FIG. 10 is a cross-sectional view of the blood vessel-leading unit shown in FIG. 1 and the first ring shown in FIG. 5;

FIGS. 11 through 20 are cross-sectional views of a blood vessel in various positions relative to the first ring and the blood vessel-leading unit shown in FIG. 10;

FIGS. 21 through 30 are cross-sectional views of another blood vessel in various positions relative to the second ring and the blood vessel-leading unit shown in FIG. 10;

FIGS. 31 through 39 are cross-sectional views of the blood vessels and the rings in various positions relative to the ring-guiding unit shown in FIG. 5.

### DETAILED DESCRIPTION OF EMBODIMENTS

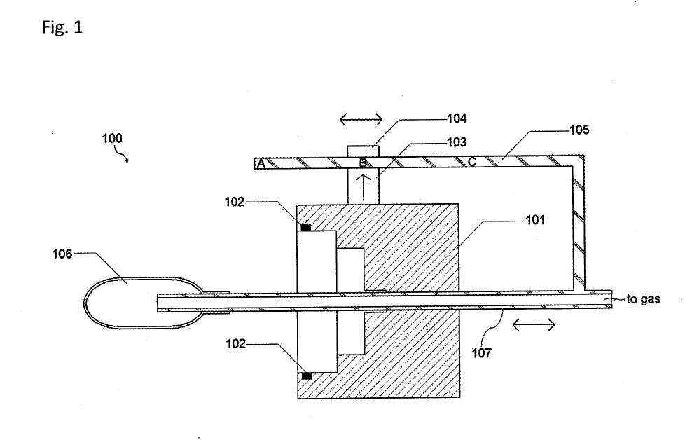

Referring to FIGS. 1 through 3, there is shown an anastomosis system in accordance with a first embodiment of the present invention. The anastomosis system includes a blood vessel-interconnecting unit 300, a ring-guiding unit and a blood vessel-leading unit 100.

Referring to FIG. 1, the blood vessel-leading unit 100 includes a ring holder 101, an elastic ring 102, an indicator 103, a knob 104, an L-shaped balloon-driving element 105, a balloon 106 and a tube 107. The ring holder 101 is in the form of a cap. The ring holder 101 includes a cavity defined therein and a channel in communication with the cavity.

The elastic ring 102 is placed in an annular groove defined in an internal face of the ring holder 101. The elastic ring 102 is made of rubber or plastics for example.

The indicator 103 is printed on or inscribed in a block fixed on the ring holder 101. The indicator 103 is an arrow head or a line for example. The indicator 103 is used together with the marks A, B and C printed on or inscribed in the balloon-driving element 105 to indicate three positions for the balloon-driving element 105 and the balloon 106.

The knob 104 is placed rotationally on the ring holder 101. The balloon-driving element 105 includes a first section extending through the knob 104 and a second section extending to the tube 107. Although not shown, there is a mechanism between the knob 104 and the first section of the balloon-driving element 105 so that by rotating the knob 104, the first section of the balloon-driving element 105 is moved forward or backward relative to the ring holder 101. The mechanism, for example, may include a thread formed on an internal face of the knob 104 and engaged with another thread formed on the first section of the balloon-driving element 105.

The tube 107 is parallel to the first section of the balloon-driving element 105. The tube 107 and the balloon-driving element 105 are preferably made one piece. The tube 107 movably extends through the channel and cavity of the ring holder 101. The tube 107 is attached to the balloon 106 at an end and attached to a gas source such as a pump at another end so that the inflation and deflation of the balloon 106 can be well controlled.

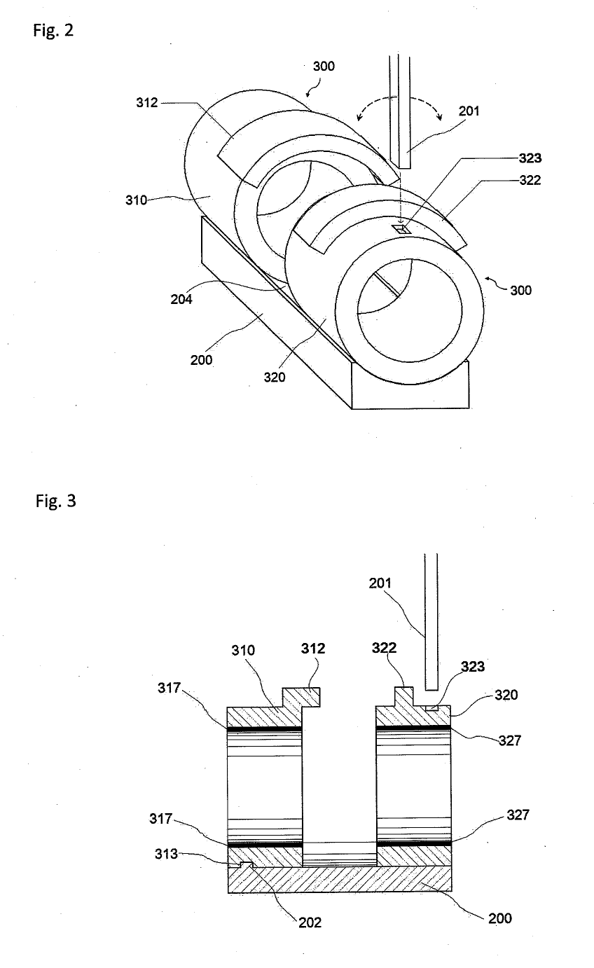

Referring to FIGS. 2 and 3, the ring-guiding unit includes a base 200 and a ring-driving element 201. The base 200 is in the form of a trough. A boss 202 is formed on the bed of a groove 204 defined in the base 200. That is, the boss 202 is formed on a concave upper face of the base 200. The ring-driving element is in the form of a rod.

The blood vessel-interconnecting unit 300 consists of two rings 310 and 320. The ring 310 includes a lip 312 extending beyond an end and a recess 313 defined in an external face. The shape and size of the recess 313 are made corresponding to the boss 202. The ring 320 includes a rib 322 formed thereon and a bore 323 defined in an external face. The rib 322 is shaped in compliance with the lip 312 to allow snap-on engagement after the alignment of the rings. The bore 323 is made corresponding to the ring-driving element 201. In anastomosis, a blood vessel is attached to the first ring 310 after the former is guided into the latter by the blood vessel-leading unit 100. Another blood vessel is attached to the second ring 320 after the former is guided into the latter by the blood vessel-leading unit 100. The rings 310 and 320 are joined together after they are guided to each other by the ring-guiding unit.

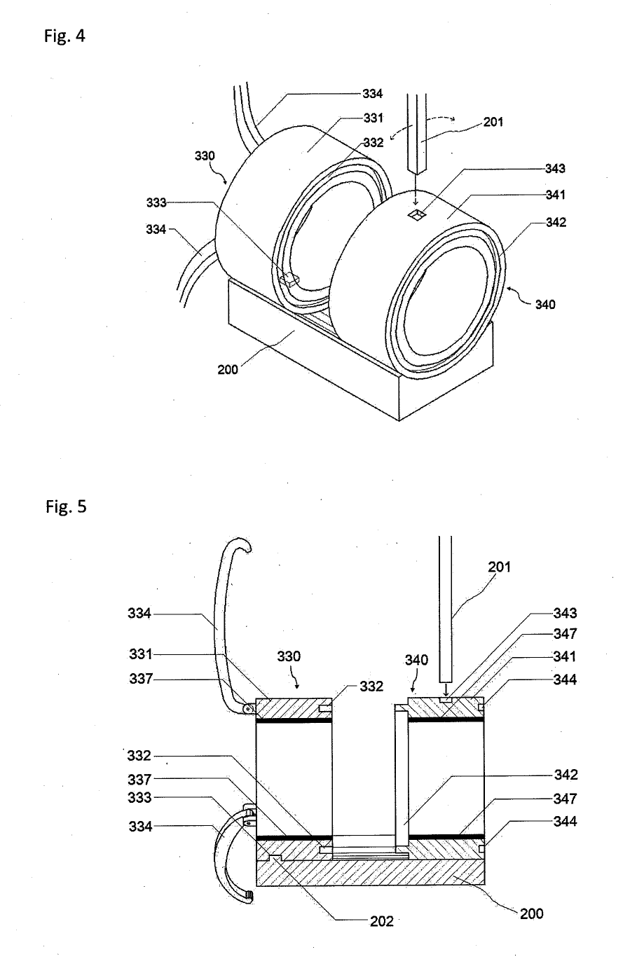

Referring to FIGS. 4 and 5, there is shown an anastomosis system in accordance with a second embodiment of the present invention. The second embodiment is like the first embodiment except including two ring sets 330 and 340 instead of the rings 310 and 320.

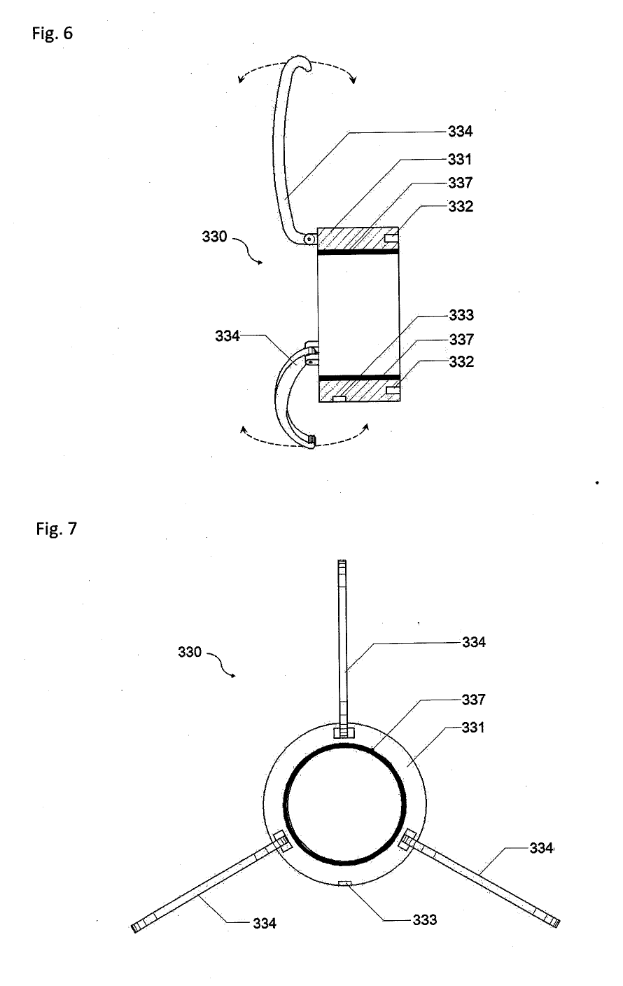

Referring to FIGS. 6 and 7, the ring set 330 includes a ring 331 and three buckles 334. Each of the buckles 334 includes a first end pivotally connected to an end of the body 331 and a second end for engagement with an end of the ring set 340. The ring 331 includes an annular groove 332 defined in another end and a recess 333 defined in an external face. The shape and size of the recess 333 are made corresponding to the boss 202.

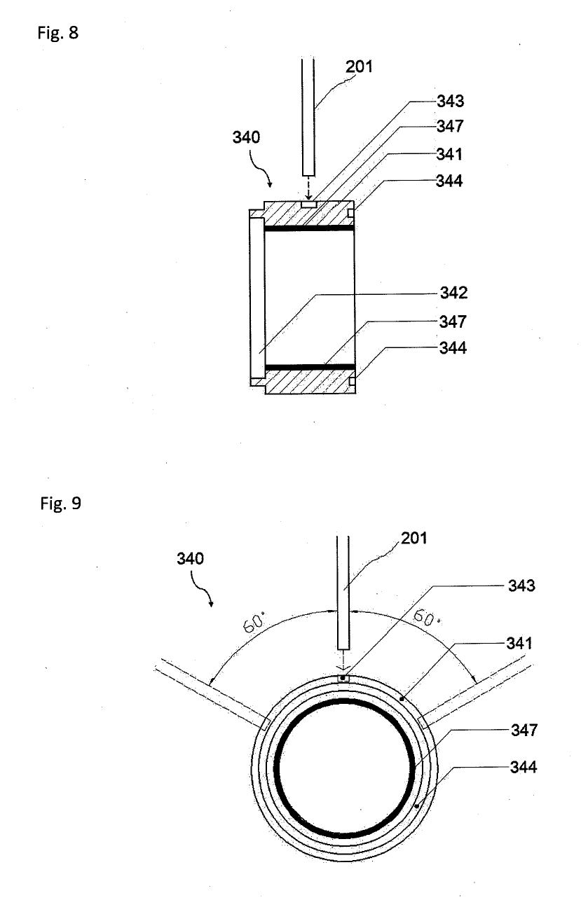

Referring to FIGS. 8 and 9, the ring set 340 includes a ring 341, an annular lip 342 extending from an end of the ring 341, an annular groove 344 defined in another end of the ring 341, and a bore 343 defined in an external face of the ring 341. The bore 343 is made corresponding to the ring-driving element 201.

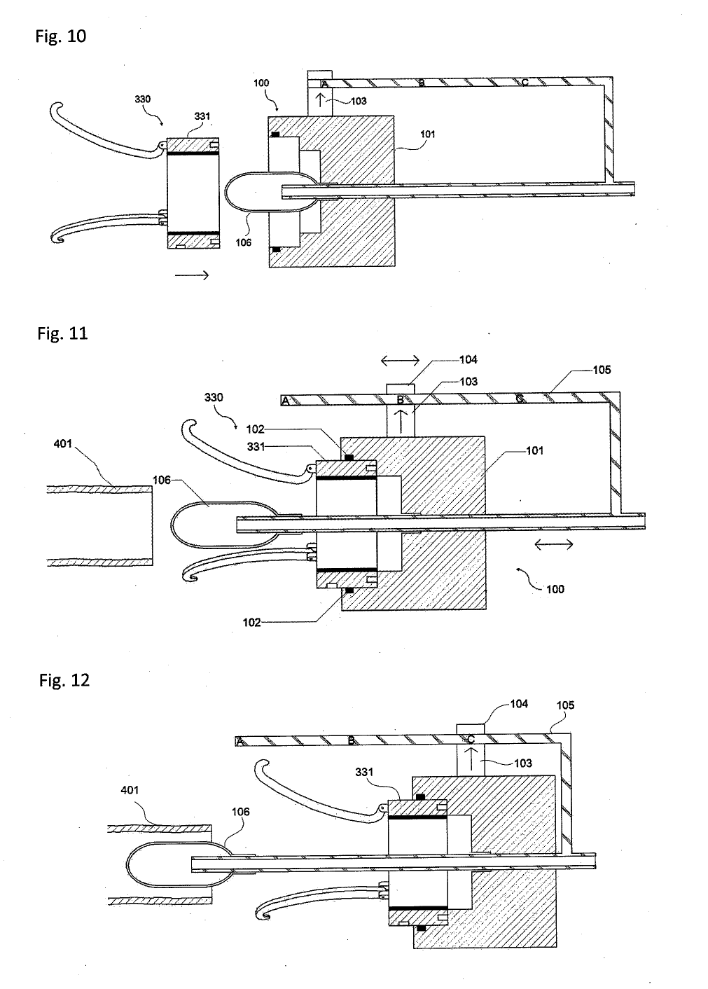

Referring to FIG. 10, there are shown the ring set 330 and the blood vessel-leading unit 100. The indicator 103 is pointed at the mark A to set the balloon 106 in a retracted position. The balloon 106 is deflated. The ring 331 is then ready to be put into the ring holder 101.

Referring to FIG. 11, there is shown a blood vessel 401 in addition to the ring set 330 and the blood vessel-leading unit 100. The ring 331 is placed partially in the ring holder 101 and held securely by the elastic ring 102. By rotating the knob 104, the indicator 103 is pointed at the mark B, and the balloon-driving element 105 and the balloon 106 are moved forward to pass through the ring 331 and access to the blood vessel 401.

Referring to FIG. 12, by rotating the knob 104, the indicator 103 is pointed at the mark C, and the balloon-driving element 105 and the balloon 106 are moved forward further to allow the insertion of the balloon 106 into the lumen of the blood vessel 401 that is placed outside the ring 331.

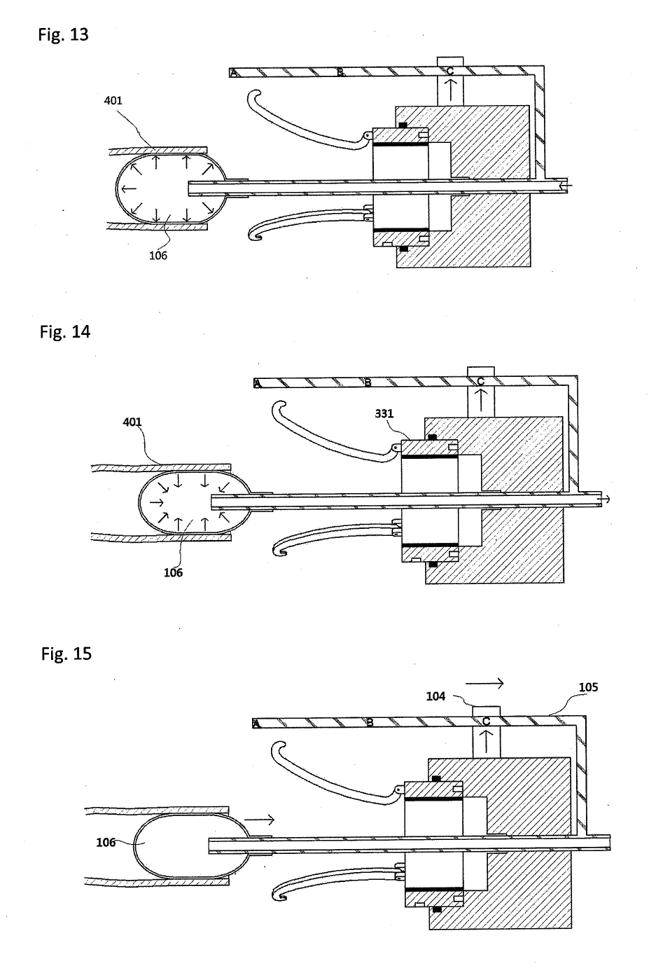

Referring to FIG. 13, the balloon 106 is inflated to bring an external face thereof into contact with an internal face of the blood vessel 401. The contact of the balloon 106 with the blood vessel 401 is firm and uniform so that the blood vessel wall is attached to the balloon well and they can be moved together.

Referring to FIG. 14, some gas is ventilated from the balloon 106 to assure the external diameter of the balloon-supported blood vessel 401 is smaller than the internal diameter of the ring 331 before the balloon 106 is retracted to pass through the ring 331.

Referring to FIG. 15, by rotating the knob 104, the balloon-driving element 105 and the balloon 106 are retracted and moved toward the ring 331, and so is the blood vessel 401.

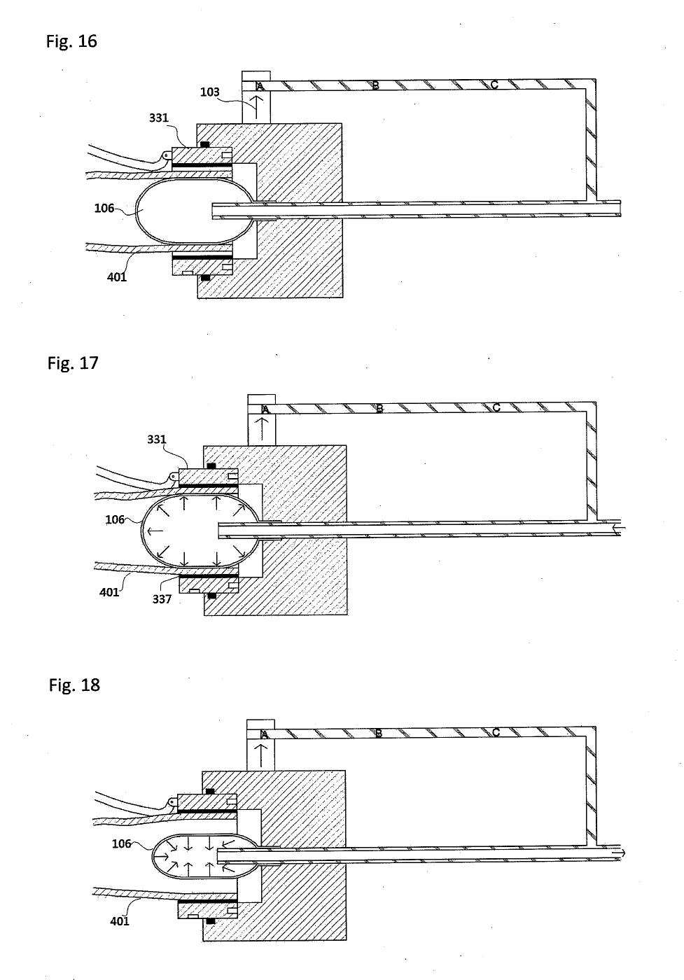

Referring to FIG. 16, the indicator 103 is pointed at the mark A. Now, the edge of the blood vessel 401 is aligned with the one end of the ring 331.

Referring to FIG. 17, the balloon 106 is further inflated to bring the blood vessel 401 into firm contact with the ring 331. Means 337 is provided between the blood vessel 401 and the ring 331 to retain the blood vessel 401 to the ring 331. The means 337 may be retention by bio-gel or by tiny needles for example.

Referring to FIG. 18, the gas is exhausted from the balloon 106. The balloon shrinks substantially and gets detached from the blood vessel 401.

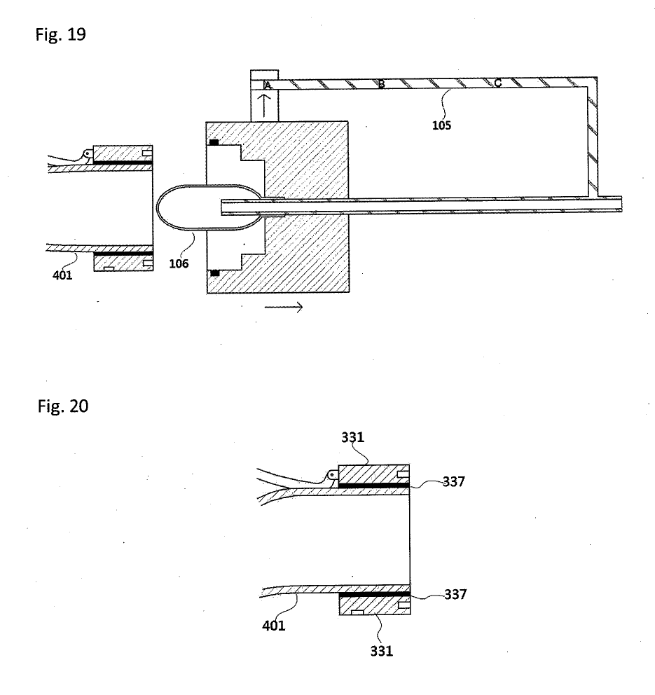

Referring to FIG. 19, by operating the balloon-driving element 105, the ring 331, which is connected to the blood vessel 401, is gently released from the ring holder 101.

Referring to FIG. 20, the blood vessel 401 is firmly attached to the ring 331 by the means 337.

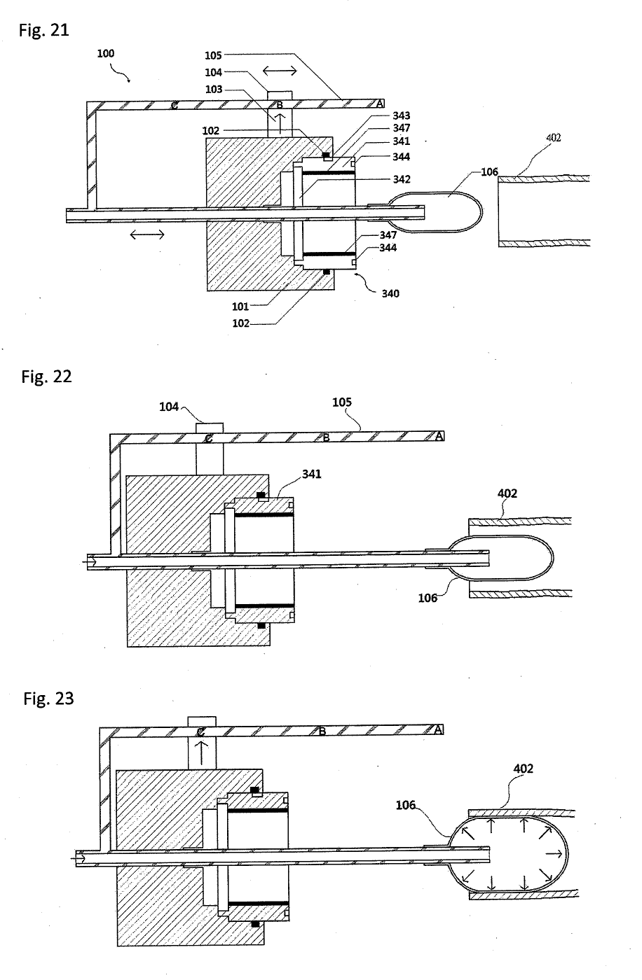

Referring to FIG. 21, there is shown a blood vessel 402 together with the ring set 340 and the blood vessel-leading unit 100. The indicator 103 is pointed at the mark B. The ring 341 is partially placed in the ring holder 101 and held securely by the elastic ring 102.

Referring to FIG. 22, by rotating the knob 104, the indicator 103 is pointed at the mark C, and the balloon-driving element 105 and the balloon 106 are moved forward to allow the insertion of the balloon 106 into the lumen of the blood vessel 402 that is placed outside the ring 341.

Referring to FIG. 23, the balloon 106 is inflated to bring an external face thereof into contact with an internal face of the blood vessel 402. The contact of the balloon 106 with the blood vessel 402 is firm and uniform so that the blood vessel wall is attached to the balloon well and they can be moved together.

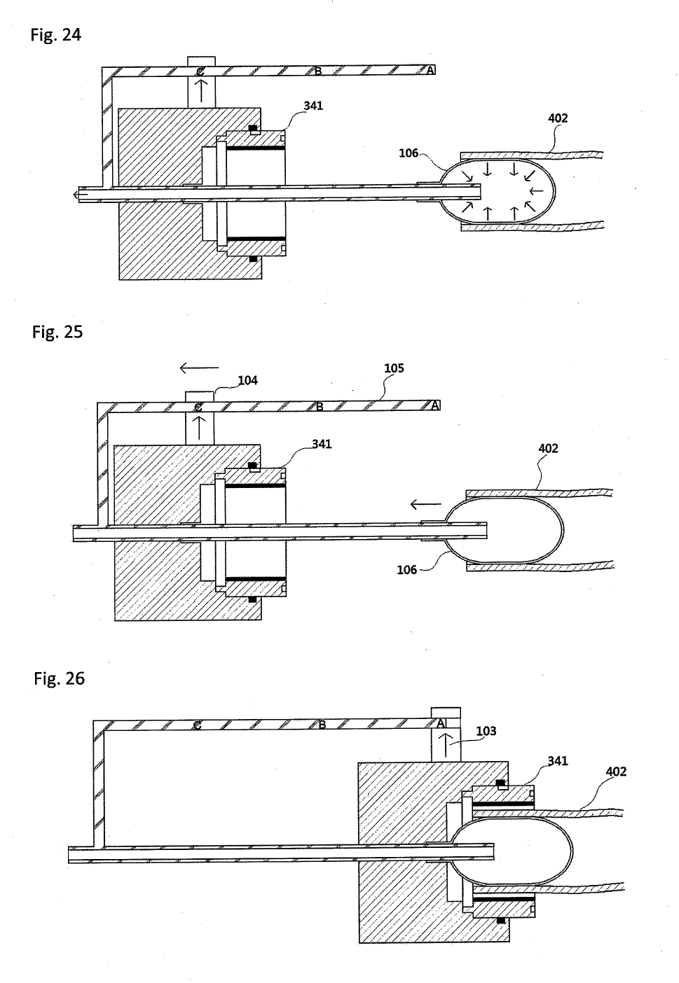

Referring to FIG. 24, some gas is ventilated from the balloon 106 to assure the external diameter of the balloon supported blood vessel 402 is smaller than an internal diameter of the ring 341 before the balloon 106 is retracted to pass through the ring 341.

Referring to FIG. 25, by rotating the knob 104, the balloon-driving element 105 and the balloon 106 are retracted and moved toward the ring 341, and so is the blood vessel 402.

Referring to FIG. 26, the indicator 103 is pointed at the mark A. Now, the edge of the blood vessel 402 is aligned with the one end of the ring 341.

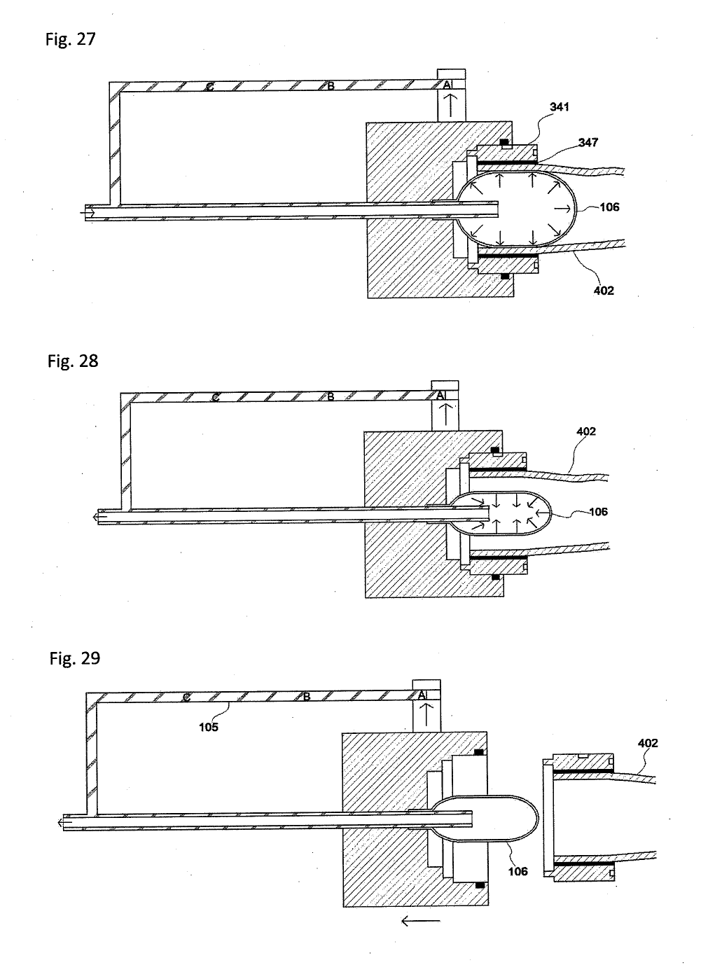

Referring to FIG. 27, the balloon 106 is further inflated to bring the blood vessel 402 into firm contact with the ring 341. Means 347 is provided between the blood vessel 402 and the ring 341 to retain the blood vessel 402 to the ring 341. The means 347 may be bio-gel or tiny needles for example.

Referring to FIG. 28, the gas is exhausted from the balloon 106. The balloon shrinks substantially and gets detached from the blood vessel 402.

Referring to FIG. 29, by operating the balloon-driving element 105, the ring 341, which is connected to the blood vessel 402, is gently released from the ring holder 101.

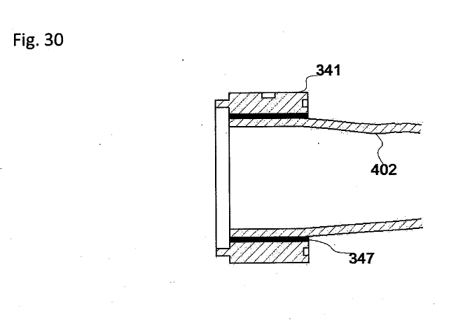

Referring to FIG. 30, the blood vessel 402 is firmly attached to the ring 341 by the means 347.

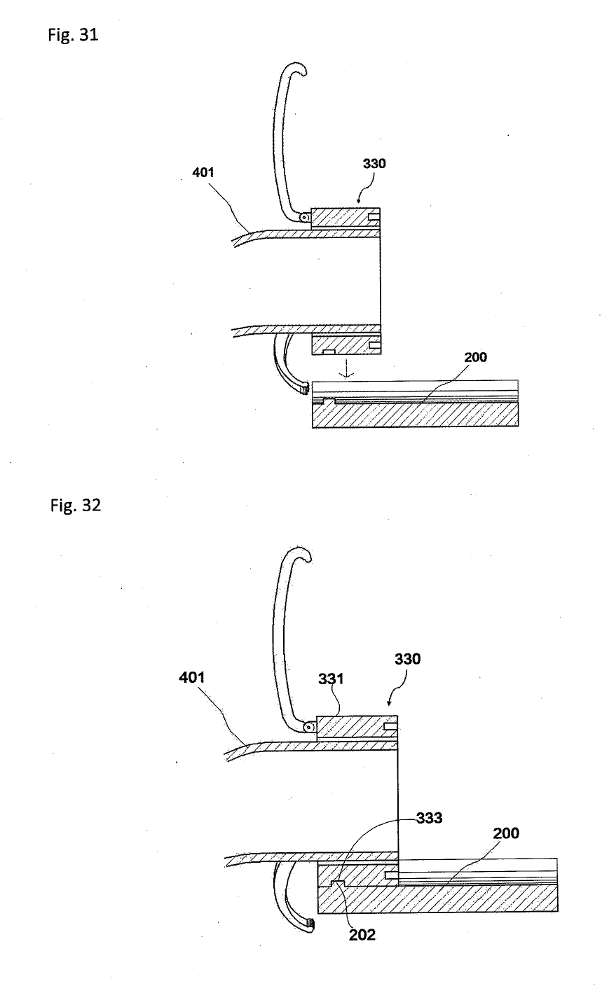

Referring to FIG. 31, the ring set 330 and the blood vessel 401 are moved toward the base 200.

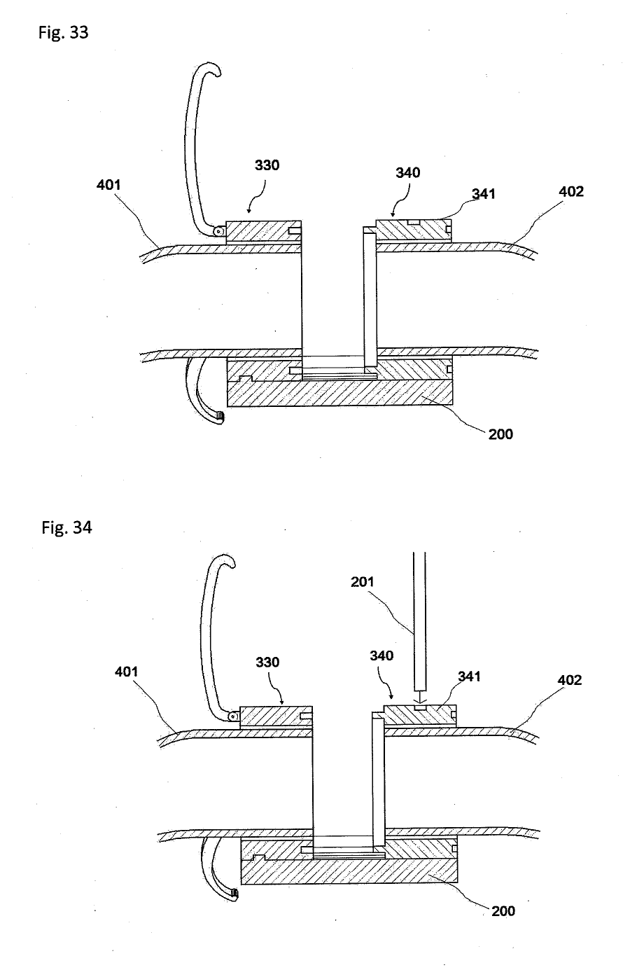

Referring to FIG. 32, the ring set 330 and the blood vessel 401 are placed on the base 200. The ring 331 is partially placed in the groove defined in the base 200, with the boss 202 placed in the recess 333 to position the ring 331 on the base 200.

Referring to FIG. 33, the ring set 340 and the blood vessel 402 are placed on the base 200. The ring 341 is partially placed in the groove defined in the base 200.

Referring to FIG. 34, the ring-driving element 201 is moved toward the ring 341.

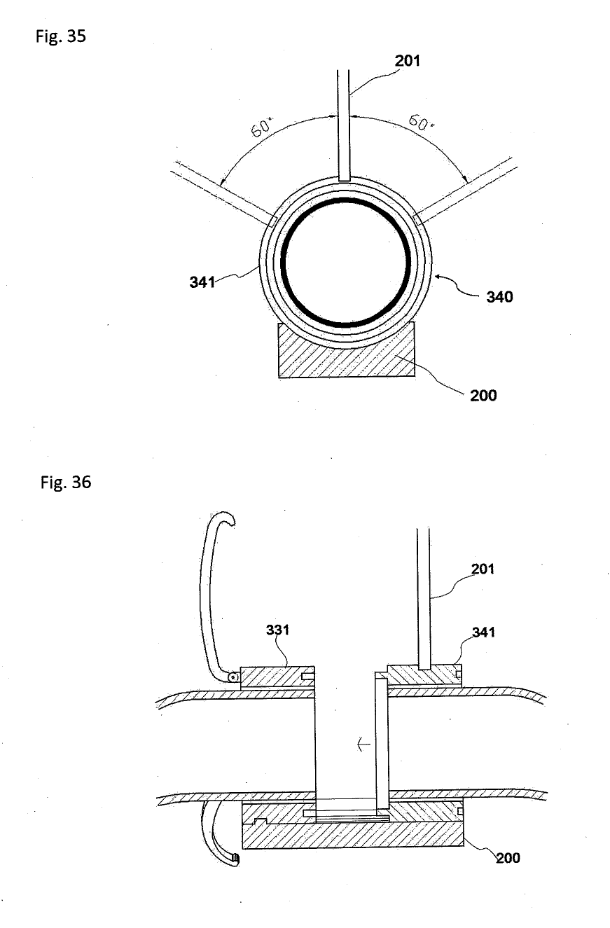

Referring to FIG. 35, a lower end of the ring-driving element 201 is inserted in the bore 343 defined in the ring 341. By operating the ring-driving element 201, the ring 341 is rotated in the groove defined in the base 200.

Referring to FIG. 36, by operating the ring-driving element 201, the ring 341 is moved toward the ring 331 in and along the groove defined in the base 200.

Referring to FIG. 37, by operating the ring-driving element 201, the ring 341 is placed against the ring 331. The annular lip 342 is placed in the annular groove 332. Thus, the axis of the ring 341 is aligned with the axis of the ring 331.

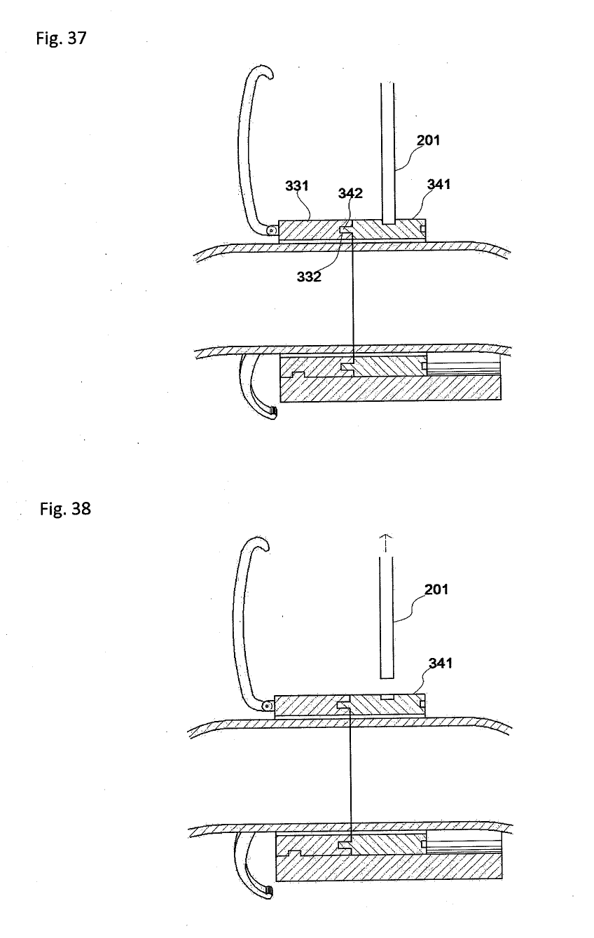

Referring to FIG. 38, the ring-driving element 201 is detached from the ring 341.

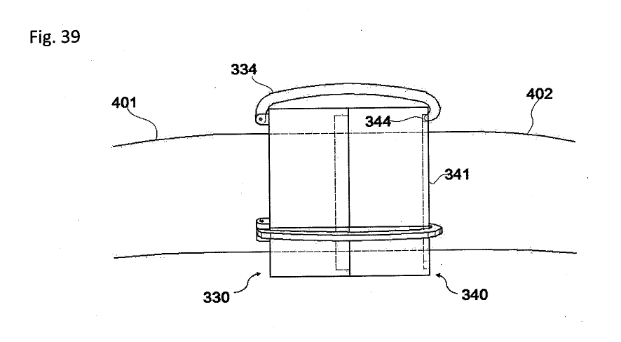

Referring to FIG. 39, the buckles 334 are engaged with the ring 341. The second end of each of the buckles 334 is inserted in the annular groove 344. Hence, the ring sets 330 and 340 are firmly connected to each other, and so are the blood vessels 401 and 402.

The anastomosis system of the present invention exhibits several advantages over the prior art. At first, the insertion and fixation of the blood vessel 401 or 402 in the ring 331 or 341 is convenient because of the use of the blood vessel-leading unit 100 to drive the balloon 106 together with the blood vessel 401 or 402.

Secondly, the rotation of the ring 341 relative to the ring 331 is efficient because only the ring 341 is rotated on the base 200 while the ring 331 is not rotated because the boss 202 is placed in the recess 333 to position the ring 331 on the base 200.

Thirdly, the movement of the ring 341 relative to the ring 331 is efficient because only the ring 341 is moved on the base 200 while the ring 331 is not moved because the boss 202 is placed in the recess 333 to position the ring 331 on the base 200.

Fourthly, the rotation and movement of the ring 341 relative to the ring 331 are efficient because of the use of the ring-driving element 201 in the bore 343.

Fifthly, the alignment of the axis of the ring 341 to the axis of the ring 331 is precise because of the insertion of the annular lip 342 in the annular groove 332.

Sixthly, the interconnection of the rings 331 and 341 is efficient because of the use of the buckles.

Seventhly, the interconnection of the rings 331 and 341 is effective because of the use of the annular groove 344 for receiving the second end of each of the buckles 334.

The present invention has been described via the detailed illustration of the preferred embodiment. Those skilled in the art can derive variations from the preferred embodiment without departing from the scope of the present invention. Therefore, the preferred embodiment shall not limit the scope of the present invention defined in the claims.

1. An anastomosis system including:
a base 200;
a first ring set 340 including a first ring 341 placed on the base 200 in a rotatable and movable manner for holding a first blood vessel 402; and
a second ring 331 including:
a second ring 331 placed on the base 200 for holding a second blood vessel 401; and
at least one buckle 334 connected to the second ring 331 for engagement with the first ring 341 so that the interconnection of the rings 330, 340 is efficient.

2. The anastomosis system according to claim 1, wherein the first ring 341 includes an annular groove 344 for receiving an end of the buckle 334 so that the interconnection of the rings 331 and 341 is effective.

3. The anastomosis system according to claim 1, wherein the first ring 341 includes an annular lip 342 extending from an end, wherein the second ring 331 includes an annular grooved 332 for receiving the annular lip 342, thus precisely aligning the axis of the first ring 341 to the axis of the second ring 331.

4. The anastomosis system according to claim 1, wherein the base 200 includes a boss 202 formed thereon, wherein the second ring 331 includes a recess 333 for receiving the boss 202 so that the rotation and movement of the first ring 341 relative to the second ring 331 is efficient because only the first ring 341 is rotated on the base 200 while the second ring 331 is not rotated.

5. The anastomosis system according to claim 1, further including a ring-driving element 201, wherein the first ring 341 includes a bore 343 for receiving an end of the ring-driving element 201 so that the ring-driving element 201 is operable to efficiently rotate and move the first ring 341 relative to the second ring 331.

6. The anastomosis system according to claim 1, further including a blood vessel-leading unit 100 for guiding the blood vessels 401, 402 into the rings 331, 341 and pressing the blood vessels 401, 402 against the rings 331, 341.

7. The anastomosis system according to claim 6, wherein the blood vessel-leading unit 100 includes:
a ring holder 101 for holding each of the rings 331, 341;
a balloon 106 for insertion in and inflation against each of the blood vessels 401, 402 so that they are movable together;
a tube 107 connected to the balloon 106 and movably inserted through the ring holder 101;
a balloon-driving element 105 connected to the tube 107 and movable on the ring holder 101 to cause the balloon 106 to lead each of the blood vessels 401, 402 into the corresponding ring 331, 341 so that each of the blood vessels 401, 402 is smoothly inserted in the corresponding ring 331, 341.

8. The anastomosis system according to claim 7, wherein the blood vessel-leading unit 100 includes a knob 104 operable to move the balloon-driving element 105.

9. An anastomosis system including:
a base 200 including a boss 202 formed thereon;
a first ring set 340 including a first ring 341 placed on the base 200 in a rotatable and movable manner for holding a first blood vessel 402; and
a second ring set 330 including:
a second ring 331 for holding a second blood vessel 401; and
a recess 333 defined in the second ring 331 for receiving the boss 202 so that the rotation and movement of the first ring 341 relative to the second ring 331 is efficient because only the first ring 341 is rotated on the base 200 while the second ring 331 is not rotated.

10. The anastomosis system according to claim 9, wherein the first ring 341 includes an annular lip 342 extending from an end, wherein the second ring 331 includes an annular grooved 332 for receiving the annular lip 342, thus precisely aligning the axis of the first ring 341 to the axis of the second ring 331.

11. The anastomosis system according to claim 9, further including a ring-driving element 201, wherein the first ring 341 includes a bore 343 for receiving an end of the ring-driving element 201 so that the ring-driving element 201 is operable to efficiently rotate and move the first ring 341 relative to the second ring 331.

12. The anastomosis system according to claim 9, further including a blood vessel-leading unit 100 for guiding the blood vessels 401, 402 into the rings 331, 341 and pressing the blood vessels 401, 402 against the rings 331, 341.

13. The anastomosis system according to claim 12, wherein the blood vessel-leading unit 100 includes:
a ring holder 101 for holding each of the rings 331, 341;
a balloon 106 for insertion in and inflation against each of the blood vessels 402;
a tube 107 connected to the balloon 106 and movably inserted through the ring holder 101; and
a balloon-driving element 105 connected to the tube 107 and movable on the ring holder 101 to cause the balloon 106 to lead each of the blood vessels 401, 402 into the corresponding ring 331, 341 so that each of the blood vessels 401, 402 is smoothly inserted in the corresponding ring 331, 341.

14. The anastomosis system according to claim 13, wherein the blood vessel-leading unit 100 includes a knob 104 operable to move the balloon-driving element 105.

15. An anastomosis system including:
a base 200;
two rings 331, 341 each for holding a blood vessel 401, 402, wherein at least one of the rings 341 is placed on the base 200 in a movable and rotational manner; and
a blood vessel-leading unit 100 for guiding the blood vessels 401, 402 into the rings 331, 341 and pressing the blood vessels 401, 402 against the rings 331, 341.

16. The anastomosis system according to claim 15, wherein the blood vessel-leading unit 100 includes:
a ring holder 101 for holding each of the rings 331, 341;
a balloon 106 for insertion in and inflation against each of the blood vessels 402;
a tube 107 connected to the balloon 106 and movably inserted through the ring holder 101; and
a balloon-driving element 105 connected to the tube 107 and movable on the ring holder 101 to cause the balloon 106 to lead each of the blood vessels 401, 402 into the corresponding ring 331, 341 so that each of the blood vessels 401, 402 is smoothly inserted in the corresponding ring 331, 341.

17. The anastomosis system according to claim 16, wherein the blood vessel-leading unit 100 includes a knob 104 operable to move the balloon-driving element 105.

18. The anastomosis system according to claim 16, wherein one of the rings 341 includes an annular lip 342 extending from an end, wherein the other ring 331 includes an annular grooved 332 for receiving the annular lip 342, thus precisely aligning the axes of the rings 331, 341 to each other.

19. The anastomosis system according to claim 16, further including a ring-driving element 201, wherein one of the rings 341 includes a bore 343 for receiving an end of the ring-driving element 201 so that the ring-driving element 201 is operable to efficiently rotate and move the rings 341, 331 relative to each other.
